(19) United States
(12) Patent Application Publication
(10) Pub. No.: US 2025/0221416 A1
(43) Pub. Date: Jul. 10, 2025

(54) CARRIER-BASED AGRICULTURAL BIOFERTILIZER COMPOSITION FOR ENHANCED DISEASE MANAGEMENT IN LEGUMES

(71) Applicants: BENOY BERRY, LONDON (GB); PRAVEEN KUMAR DIVVELA, WEST GODAVARI (IN)

(72) Inventors: BENOY BERRY, LONDON (GB); PRAVEEN KUMAR DIVVELA, WEST GODAVARI (IN)

(21) Appl. No.: 19/091,940

(22) Filed: Mar. 27, 2025

(30) Foreign Application Priority Data
Mar. 30, 2024 (GB) GB2404620.3

(51) Int. Cl. A01N 63/27; A01N 25/08; A01P 21/00; C12N 1/14

(52) U.S. Cl. CPC A01N 63/27; A01N 25/08; A01P 21/00; C12N 1/14

(57) ABSTRACT

Exemplary embodiments of the present disclosure are directed towards a carrier-based agricultural biofertilizer composition for enhanced disease management in legumes includes, an isolated fluorescens strain CGA FPBPF-034 having the deposit accession number KY495220, and an agriculturally acceptable solid or liquid formulation with an organic or inorganic stabilizer to provide stability and support for the Pseudomonas species, thereby enhancing the disease management efficacy and overall health of the plants.

### CROSS-REFERENCE TO RELATED APPLICATIONS

This patent application claims priority benefit of UK Patent Application No: GB2404620.3, entitled “A CARRIER-BASED AGRICULTURAL BIOFERTILIZER COMPOSITION FOR ENHANCED DISEASE MANAGEMENT IN LEGUMES”, filed on 30 Mar. 2024. The entire contents of the patent application are hereby incorporated by reference herein in its entirety.

### TECHNICAL FIELD

The present disclosure generally relates to microbial biofertilizer compositions, more particularly, the invention relates to a carrier-based agricultural biofertilizer composition for enhanced disease management in legumes.

### BACKGROUND

Agriculture stands as a foundational pillar of human civilization, providing sustenance, economic livelihoods, and raw materials for numerous industries. Yet, agriculture faces persistent challenges, and one of the most formidable is the ongoing battle against crop diseases, particularly fungal diseases. These afflictions have a long history of causing reduced yields, compromised crop quality, and significant economic losses for farmers.

Fungal diseases in crops have a substantial impact on global food production and quality, affecting a wide range of crops, including cereals, vegetables, fruits, and legumes. Among these, Cowpea (Vigna unguiculata) and Groundnut (Arachis hypogea) hold paramount importance. Cowpea is a vital protein source, especially in regions with limited access to animal protein, while Groundnut serves as a primary source of edible oil and protein worldwide.

Scab disease, caused by the fungal pathogen Sphaceloma sp., poses a particularly menacing threat to Cowpea crops. This disease results in distinct lesions on Cowpea pods, leaves, and stems, leading to severe yield losses and compromised crop quality. Late Leaf Spot disease, on the other hand, severely affects Groundnut crops due to the causative agent, Cercospora sp., which causes dark blemishes on Groundnut plant foliage, leading to reduced yields.

The urgent need for sustainable solutions has led to research into Biological Control Agents (BCAs), living organisms like bacteria, fungi, viruses, and nematodes, which can mitigate the impact of plant pathogens. Among BCAs, certain strains of Pseudomonas fluorescens, a naturally occurring bacterium, have shown immense promise in suppressing fungal diseases and promoting plant growth.

Pseudomonas fluorescens CGA FPBPF-034 is one such strain with remarkable potential, known for its beneficial properties in agriculture. This Gram-negative bacterium exhibits antagonistic behavior against a broad spectrum of fungal pathogens. However, despite its promise, there is a significant gap in the prior art concerning its specific evaluation against Scab disease in Cowpea and Late Leaf Spot disease in Groundnut. Comprehensive exploration and documentation are essential for its development and application as a tailored biofertilizer for these specific crops and diseases.

Conventional approaches to managing fungal diseases in crops, historically reliant on synthetic chemical fungicides, have faced substantial drawbacks. These chemical interventions, while providing a degree of effectiveness, have engendered a host of environmental and health concerns. Notably, the indiscriminate use of chemical fungicides has led to the emergence of fungicide-resistant pathogen strains, posing a grave and persistent threat to long-term disease management efforts. Moreover, the presence of chemical residues in harvested crops has raised significant apprehensions regarding food safety and its potential implications for human health. The runoff of these agricultural chemicals into water sources further exacerbates these concerns, endangering aquatic ecosystems. As such, there is a pressing need to explore alternative, sustainable, and environmentally conscious methods for combatting fungal diseases in agricultural settings.

In the light of the aforementioned discussion, there exists a need for a certain biofertilizer formulation with novel and improved methodologies that would overcome the above-mentioned disadvantages.

### SUMMARY

The following presents a simplified summary of the disclosure in order to provide a basic understanding of the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

An embodiment of the present disclosure is directed towards a carrier-based agricultural biofertilizer composition.

An objective of the present disclosure is directed towards introducing an innovative method for assessing the fungicidal properties of Pseudomonas fluorescens CGA FPBPF-034 against economically significant fungal diseases in Cowpea and Groundnut, filling a void in the prior art.

An objective of the present disclosure is directed towards providing a detailed evaluation procedure, encompassing various steps, which facilitates a comprehensive assessment of Pseudomonas fluorescens CGA FPBPF-034's efficacy in controlling Scab disease on Cowpea and Late Leaf Spot disease on Groundnut.

An objective of the present disclosure is directed towards the development of a unique composition that enhances the adherence and survival of Pseudomonas fluorescens CGA FPBPF-034 on plant surfaces, maximizing its biocontrol properties, and promoting environmentally friendly and sustainable agricultural practices.

An objective of the present disclosure is directed towards extending the invention's applications to diverse agricultural settings, including field cultivation, greenhouse practices, and organic farming, offering versatile and effective disease management solutions that enhance crop yield, quality, and overall agricultural sustainability.

An objective of the present disclosure is directed towards positioning the invention at the intersection of agriculture, microbiology, and plant pathology, addressing the pressing need for sustainable disease management in agriculture by harnessing the potential of Pseudomonas fluorescens CGA FPBPF-034 as a biocontrol agent, providing a holistic approach to combat fungal diseases in Cowpea and Groundnut.

An objective of the present disclosure is directed towards providing a carrier-based agricultural biofertilizer composition that incorporates Pseudomonas fluorescens CGA FPBPF-034, a bacterium with a deposit accession number KY495220, offering a potent and specific tool for enhanced disease management in legumes.

An objective of the present disclosure is directed towards a composition that harnesses Pseudomonas fluorescens CGA FPBPF-034 strain, isolated from the rhizosphere and rhizoplane in Yola, Nigeria, tapping into its natural potential for combating legume fungal diseases.

An objective of the present disclosure is directed towards the development of agriculturally acceptable solid and liquid formulations, integrated with a stabilizer, which collectively ensure the stability and viability of the Pseudomonas species within the composition.

An objective of the present disclosure is directed towards a method of isolating Pseudomonas fluorescens CGA FPBPF-034 through a serial dilution technique and spread plate method, ensuring its purity and efficacy as a biocontrol agent.

An objective of the present disclosure is directed towards a solid formulation of the composition that employs agriculturally acceptable carriers, such as talc, vermiculite, peat, and compost, providing sustained release and growth support for Pseudomonas fluorescens CGA FPBPF-034 in the soil, enhancing its long-term disease management effects.

An objective of the present disclosure is directed towards a composition that leverages the multifunctional attributes of Pseudomonas fluorescens CGA FPBPF-034, including its ability to inhibit fungal plant pathogens, produce hydrolytic enzymes, and secrete Siderophore, contributing to improved plant health, root development, and overall vigor.

[symbol] objective of the present disclosure is to promote long-term soil health and fertility by leveraging the biofertilizer composition, which is fortified by an agriculturally acceptable solid and liquid formulation, leading to the enrichment of soil microbiota and structure over time.

An objective of the present disclosure is directed towards a method for preparing the composition, involving nutrient medium preparation, fermentation, biomass cultivation, and formulation steps, ensuring a consistent and effective biofertilizer for legume disease management.

An objective of the present disclosure is directed towards utilizing a nutrient medium with suitable carbon and nitrogen sources, such as sucrose, glucose, malt extract, and peptone, to optimize the growth conditions for Pseudomonas fluorescens CGA FPBPF-034 strain.

An objective of the present disclosure is directed towards a solid substrate fermentation method that incorporates agricultural by-products like rice bran and wheat straw, enhancing the biomass production of Pseudomonas fluorescens CGA FPBPF-034 strain in an environmentally friendly manner.

An objective of the present disclosure is directed towards a versatile application method for the biofertilizer composition, involving spraying onto foliage or root zones, ensuring uniform coverage and efficient uptake by leguminous crops, leading to improved disease resistance and enhanced fertility of the soil.

An objective of the present disclosure is directed towards treating diseases in leguminous crops.

Furthermore, the objects and advantages of this invention will become apparent from the following description and the accompanying annexed drawings.

### BRIEF DESCRIPTION OF THE DRAWINGS

In the following, numerous specific details are set forth to provide a thorough description of various embodiments. Certain embodiments may be practiced without these specific details or with some variations in detail. In some instances, certain features are described in less detail so as not to obscure other aspects. The level of detail associated with each of the elements or features should not be construed to qualify the novelty or importance of one feature over the others.

FIG. 1 is a flowchart depicting a method for preparing a carrier-based agricultural biofertilizer agricultural biofertilizer composition for enhanced disease management in legumes.

### DETAILED DESCRIPTION OF EXAMPLE EMBODIMENTS

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced item. Further, the use of terms “first”, “second”, and “third”, and the like, herein do not denote any order, quantity, or importance, but rather are used to distinguish one element from another.

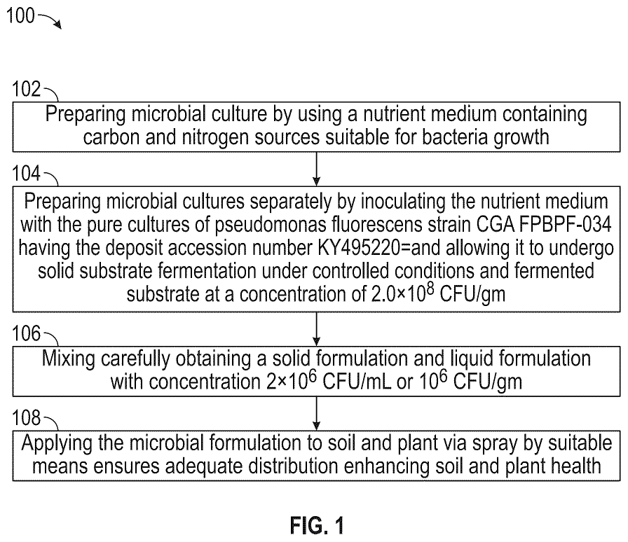

Referring to FIG. 1, FIG. 100 is a flowchart depicting a method for preparing a carrier-based agricultural biofertilizer agricultural biofertilizer composition for enhanced disease management in legumes. The method commences at step 102, by preparing microbial culture by using a nutrient medium containing carbon and nitrogen sources suitable for bacteria growth. At step 104, preparing microbial cultures separately by inoculating the nutrient medium with the pure cultures of Pseudomonas fluorescens strain CGA FPBPF-034, having the deposit accession number KY495220, =and allowing it to undergo solid substrate fermentation under controlled conditions and fermented substrate at a concentration of 2.0×10\hat{ }8 CFU/gm. Mixing carefully obtaining a solid formulation and liquid formulation with concentration 2×10\hat{ }6 CFU/mL or 10\hat{ }6 CFU/gm at step 106. At step 108, applying the microbial formulation to soil and plant via spray by suitable means ensures adequate distribution, enhancing soil and plant health.

In an embodiment of the present disclosure, to maximize the efficacy Pseudomonas fluorescens as a biocontrol agent, it may be imperative to develop a formulation that may incorporates carrier materials that may enhance its viability, dispersal, and activity within the target environment. Below is a detailed description of how the optimal carrier formulation may facilitate Pseudomonas fluorescens in potentially acting as a biocontrol agent.

Selection of Carrier: The selection of an appropriate carrier material holds paramount importance. The carrier should offer protection and stability to the cells of Pseudomonas fluorescens during storage and application. Commonly employed carrier materials encompass peat, vermiculite, clay minerals, alginate beads, and various organic materials.

The most suitable carrier material may possess the following attributes:
Porosity: A carrier that may exhibit commendable porosity may enable aeration and water retention, potentially establishing a favorable microenvironment for bacterial growth and survival.
Biodegradability: A carrier that may naturally degrade over time may be advantageous, as it may prevent accumulation within the environment.
Adhesion: It may be enabling the bacteria to adhere to its surface, thus potentially preventing wash-off subsequent to application.
Nutrient Release: Certain carriers may have the capability to gradually release nutrients, thereby providing a continuous source of nourishment for Pseudomonas fluorescens.
Augmented Bacterial Survival: The carrier may act as a protective shield, potentially safeguarding Pseudomonas fluorescens from harsh environmental conditions such as UV radiation, desiccation, and temperature fluctuations. This protective function may ensure the potential viability of the biocontrol agent until it reaches its intended target.
Enhanced Dispersion: The carrier may facilitate the even dispersion of Pseudomonas fluorescens within the designated area, which could be crucial for effective biocontrol, as it may ensure that the bacteria are in close proximity to potential pathogens.
Extended Shelf Life: Appropriate formulation with the carrier may have the potential to extend the shelf life of the biocontrol product. This extension may be indispensable for commercial viability and ease of distribution.
Compatibility with Other Products: The carrier formulation may be designed to be compatible with other agricultural inputs, such as fertilizers and pesticides, that may be utilized in conjunction with the biocontrol agent.

Suitability for Different Application Methods: The carrier formulation may be adaptable to various application methods, including seed coating, soil drenching, foliar spraying, and irrigation.
Environmentally Friendly: An ideal carrier formulation may be characterized by its environmentally friendly nature, potentially devoid of any introduction of harmful chemicals or substances into the ecosystem.
Field Testing: Before proceeding with commercial implementation, it may be advisable for the carrier formulation to undergo rigorous field testing. This testing may serve to evaluate its effectiveness in potentially controlling specific plant pathogens under real-world conditions.
Regulatory Approval: Depending on the region and intended usage, it may be a requirement for the carrier formulation and its associated Pseudomonas fluorescens strain to seek regulatory approval. This process may involve the demonstration of safety and efficacy through scientific studies and field trials.
Monitoring and Optimization: Continuous monitoring and potential optimization of the carrier formulation may be indispensable. This process may be necessary to adapt to changing environmental conditions, pathogens, and evolving farming practices.

In an embodiment of the present disclosure, the biofertilizer composition may potentially contain biologically active chemicals that could be utilized for treating plant fungal illnesses, such as diseases like “Scab and late leaf spot.” Typically, the composition may consist of a carrier, commonly talc powder and carboxy methyl cellulose, along with bacterial biomass chosen for their potential effectiveness in maintaining plant health or potentially controlling diseases. A fungicidally effective quantity of the biocontrol formulation may be employed in the biofertilizer composition. The preparation process may involve cultivating the selected bacteria to enhance their potential survival, blending the biomass, mixing it with the carrier, and subsequently desiccating the mixture. Following activation, the dried preparation may be applied to soil, nearby growing substrate, or used as a seed coating. The bacterial seed coating may potentially assist in combating specific plant diseases or enhancing overall plant health.

In an embodiment of the present disclosure, the formulation, which may consist of Pseudomonas fluorescens strain CGA FPBPF-034, talc powder, and a CMC (Carboxymethyl cellulose) based carrier, is designed with the potential to enhance seed germination and potentially protect plants from pathogenic infections. This formulation may be applied during planting or transplanting to initiate a sequence of coordinated mechanisms that may provide effective seed germination and potential plant protection. Upon application, the CMC-based carrier may facilitate the rehydration and colonization of Pseudomonas fluorescens in the soil. In the soil environment, Pseudomonas fluorescens may potentially actively compete with soil-borne pathogens for essential nutrients, produce antimicrobial compounds, form protective biofilms on plant roots, and potentially inhibit pathogen growth. Upon uptake by the growing plant, Pseudomonas fluorescens may potentially establish systemic resistance, stimulate the production of growth-promoting compounds, sequester iron, actively suppress root diseases, enhance nutrient cycling, and potentially detoxify harmful compounds. These potentially well-coordinated mechanisms may collectively lead to improved seed germination and potentially strengthened plant defenses against pathogens, resulting in enhanced plant health, potentially reduced disease risk, and potentially increased crop yields.

These requirements may potentially find satisfactory fulfillment through the delivery method as described. The biocontrol bacteria may have the potential to rapidly and thoroughly colonize the root system and rhizosphere of the diseased plant. The desiccated product may be sprinkled directly onto the plant and soil or soilless mix intended for seed or seedling planting, or it may potentially be mixed in. When utilizing the biocontrol formulation as a soil drench, it may be advisable to consider mixing it with formic acid, even if it remains technically feasible. Alternatively, water may be used with the substance to create a straightforward aqueous solution. The substance may also hold utility as a seed covering. Regardless of the method chosen, once the soil and biocontrol product are hydrated, the spores may potentially commence germination in the soil and may act as biocontrol agents on the plant.

In an exemplary embodiment, an isolated Pseudomonas fluorescens strain CGA FPBPF-034 may be derived from the rhizosphere soil of Yola, Nigeria. This strain may have been incorporated into a biofertilizer formulation that may be designed to significantly biocontrol Scab disease on Cowpea and late leaf spot disease in Groundnut during both rainy and dry seasons. The chosen strain may demonstrate its efficacy in effectively controlling diseases and promoting plant growth and overall plant health by inducing plant systemic resistance.

Morphological Characteristics of the Pseudomonas fluorescens strain CGA FPBPF-034:
Cell Shape and Size: Typically rod-shaped with dimensions of 0.78 μm width and 1.9 μm length.
Gram-Negative Staining: Exhibits Gram-negative characteristics with a thin peptidoglycan layer and outer membrane.
Motility: Often motile due to polar flagella, facilitating movement in liquid and on surfaces.
Colony Morphology: Colonies on solid media are circular, smooth, and convex with a shiny, iridescent appearance, often greenish-yellow or fluorescent.
Fluorescent Pigments: Produces fluorescent pigments like pyoverdine and pyocyanin, visible under UV light.
Biofilm Formation: Capable of forming biofilms, aiding surface adherence and diverse applications.
Exopolysaccharides (EPS): Produce EPS involved in biofilm formation, protection, and environmental interactions.
Flagellar Arrangement: A single polar flagellum responsible for motility.

Amplification and 16S rRNA Gene Sequence Analysis: Following the potential amplification of the partial 16S rRNA gene, cycle sequencing may have been carried out through MACROGEN in Korea. The resulting amplified product might have undergone sequencing utilizing the forward sequencing reaction mix. To establish potential homology, the DNA sequence may have been queried using the BLAST search engine at the NCBI site (ncbi.nlm.nih.gov) and FASTA (ebi.ac.uk). The FASTA homology search may have revealed similarity to Pseudomonas fluorescens strain CGA FPBPF-034, and the corresponding strain obtained from NCBI may have been designated as KY495220.

In an exemplary embodiment of the present disclosure, a fed-batch fermentation procedure may potentially be utilized to cultivate the bacterial inoculum. The process may continue until a substantial cell biomass, which typically may exceed 10\hat{ }8 CFU per ml, may be achieved. Once the desired cell biomass is potentially attained, it may be harvested. In an ideal scenario, this cell mass may undergo a concentration step, potentially eliminating the need to maintain both the culture media and the cell mass separately. This concentration step may serve to potentially increase the biocontrol agent's concentration while also simplifying the overall processes. Before being incorporated into the talc powder, the moist or wet biomass may be homogenized or blended for uniformity.

In another exemplary embodiment of the present disclosure, the biofertilizer formulation may potentially be prepared by combining talc powder and the cell biomass, with the possible addition of CMC as a stabilizing agent. In liquid formulations, distilled water may be used, potentially excluding cell biomass, while in granular formulations, the spores might be enclosed within organic granules.

According to one exemplary embodiment of the present disclosure, talc powder and CMC may potentially be selected as carrier materials in the biofertilizer formulation due to their potential ability to effectively foster bacterial growth and potential compatibility with the plant environment. The biocontrol component of the formulation might be preferably formulated using talc powder and CMC, with commercially available wettable powder talc being a potential preferred option. To ensure the suitability of talc powder, the preparation of the biofertilizer formulation may involve measures that could eliminate microbiological contamination, such as prolonged heating or irradiation at temperatures that could exceed 100° C. to 110° C. Additionally, chemical sterilization may also be contemplated as an option to potentially inhibit fungus growth.

According to one exemplary embodiment of the present disclosure, the biomass/acid mixture may potentially be incorporated into the talc powder and CMC mixture using a chosen method. This approach may have the potential to obviate the requirement for sterile conditions when generating biomass with CMC and talc powder. While sterile facilities may remain crucial during biomass culture, conventional techniques for mixing, measuring, and transporting the biomass-talc powder and CMC combination may allow for non-sterile manipulation. This could potentially simplify the procedures for handling and preparation.

The biomass/acid mixture may be incorporated into the CMC and talc powder at a potential ratio of 0.50%: (2×108 CFU/gm), with 99.00% being talc powder and 0.50% being Carboxy methyl cellulose. The biomass, CMC, and talc powder may be comprehensively blended. This resulting mixture could be gradually dehydrated until the overall volatile content may decrease to less than 10.0%, preferably aiming for a reduction below 2.0%. Diminished levels of volatile substances may have the potential to impede the growth of bacteria and fungi, potentially contributing to the preservation of the biocontrol preparation. The emphasis on achieving maximum dryness may be preferred to potentially minimize growth. For instance, air drying after the application of thin layers (approximately 2.0 cm thick) might be considered a suitable approach. The desiccated biocontrol product may potentially remain stable for a minimum of six weeks, potentially ensuring its shelf life. The dehydrated bacterial product may appear as smooth, pale texture, and uniformly dispersed particles offer efficient application. Depending on the characteristics of the fungus, particles typically may have dark color and solid substrates.

The formulation of the biofertilizer might potentially include a sufficient quantity of bacterial cell biomass, which may exhibit a viable spore count of at least approximately 106 colony-forming units (CFU) per gram of the dried product biomass. The CFU can serve as a metric for assessing the potential viability of the spore preparation. A range spanning approximately 104 to approximately 1010 CFU per gram may be considered useful, with a preference leaning toward around 106 to approximately 108 CFU per gram. It's noteworthy that the desired cell count of the dried material could vary, contingent upon the anticipated dosage to be utilized in the soil or liquid mix, or on seeds that might be conveniently applied to the affected area of the plant following disease treatment and soil incorporation prior to planting.

According to one exemplary embodiment of the present disclosure, he dried biocontrol agents may potentially be applied to the plant, either via spray or drench to the soil, and/or incorporated into the soil where the plants to be protected by the biocontrol agent are expected to grow. Plant seeds may potentially be coated with the dried sporulated biomass product. For instance, the seeds could be rotated with the dried material in a viscous substance like carboxymethyl cellulose (CMC) until they are coated. The biofertilizer formulation might aid in potentially activating the fungal spores on the carrier, thus allowing the carrier/biomass formulation, when introduced into the soil or plant environment, to potentially facilitate the growth and proliferation of the biocontrol agent under natural conditions. After introducing the biofertilizer formulation into the soil or plant environment, the effectiveness of the formulation may be assessed by measuring the decrease in pathogen inoculum density and the prevention or reduction of soilborne disease. The use of the biofertilizer formulation may potentially reduce or prevent diseases such as scab (caused by Sphaceloma sp.) in cowpea (Vigna unguiculata) and late leaf spot (caused by Cercospora sp.) in groundnut (Arachis hypogaea).

In another exemplary embodiment, specific strains of advantageous bacteria may not naturally occur in a given field soil or, if present, may exist in limited numbers or exhibit reduced activity, thereby failing to impart any beneficial effects on plants in an unaltered or unenhanced rhizosphere. This may be true regardless of the inherent valuable traits possessed by these biocontrol bacteria. In order for a bacterial strain with inherent beneficial traits, to exert a positive impact on plant disease management, it may possess a competitive advantage and be a robust colonizer within the rhizosphere and plant tissues during active plant growth. It may be noted that without modifications or enhancements, it may be highly improbable for any native or naturally occurring fungi, including the bacterial strain Pseudomonas fluorescens CGA FPBPF-034, to confer benefits to plant health. This may highlight the importance of creating conditions that favor the colonization and effectiveness of this bacterial strains.

In another exemplary embodiment, Pseudomonas fluorescens CGA FPBPF-034 has the potential to be acquired from the rhizosphere and may exhibit the advantageous characteristic of plant disease resistance, thereby augmenting plant growth. Additionally, laboratory cultivation techniques may be employed to optimize the growth and density of the CGA FPBPF-034 population, thereby maximizing its effectiveness. These specific growth conditions can be identified to enhance the competitive advantage of Pseudomonas fluorescens CGA FPBPF-034 when administered to the rhizosphere and applied to the plant, resulting in a positive influence on plant growth. Essentially, the identification of growth conditions that may facilitate the successful colonization of the rhizosphere by CGA FPBPF-034 may enable its beneficial impact on plant growth, which would be unattainable under normal circumstances.

In another exemplary embodiment, a pure biological culture of Pseudomonas fluorescens CGA FPBPF-034 may be cultivated to create a reserve culture. Samples of this reserve culture may be stored in cryogenic vials at a temperature of −80° C. For production purposes, the frozen reserve culture may be used to introduce CGA FPBPF-034 into a flask containing nutrient broth media under specified conditions.

In another exemplary embodiment, the fungal culture may be cultivated at a temperature range of 30-32° C. during the rainy season, and alternatively, at a temperature range of 48-50° C. during the dry season. The flask culture may then be scaled up in a fermenter under similar growth conditions, resulting in an increased population and enhanced plant growth-promoting properties of Pseudomonas fluorescens CGA FPBPF-034 as the culture reaches the early stationary phase. Samples from the early stationary phase culture may be aseptically packaged in sterilized plastic bags. The final product may have a minimum concentration of the active ingredient, with a viability of at least 3×106 colony forming units per mL. The fermented culture of strain CGA FPBPF-034 may be grown under optimized conditions to ensure maximum bacterial viability and retention of its efficacy as a biocontrol agent. The formulation of Pseudomonas fluorescens stored in sterile bags may be preserved for analysis of viable counts. Notably, Pseudomonas fluorescens CGA FPBPF-034 exhibits a response to temperature variation when subjected to different temperatures. Shelf-life studies may indicate that even after 18 months of storage at both temperatures, the minimum count of bacterial cells remains at 2×106, demonstrating the sustained viability of the strain.

According to one exemplary embodiment of the present disclosure, Pseudomonas fluorescens strain CGA FPBPF-034 may be isolated from the rhizosphere and rhizoplane from Yola, Nigeria.

In another exemplary embodiment of the present disclosure, Pseudomonas fluorescens strain CGA FPBPF-034 was isolated by a serial dilution technique and spread plate method.

According to one exemplary embodiment of the present disclosure, the agriculturally acceptable solid formulation comprises carriers selected from the group consisting of talc, vermiculite, peat, and compost, providing a suitable matrix for sustained release and growth of Pseudomonas fluorescens strain CGA FPBPF-034.

According to one exemplary embodiment of the present disclosure, Pseudomonas fluorescens strain CGA FPBPF-034 has the ability to reduce the growth of fungal plant pathogens in vitro and can produce hydrolytic enzymes and Siderophore, thereby regulating plant disease and growth, improving root development, and enhancing overall plant vigor.

According to one exemplary embodiment of the present disclosure, wherein the nutrient medium comprises a combination of carbon and nitrogen sources, selected from the group consisting of sucrose, glucose, malt extract, and peptone, to provide an optimal growth environment for Pseudomonas fluorescens strain CGA FPBPF-034.

According to one exemplary embodiment of the present disclosure, the controlled fermentation conditions in step 2 comprise maintaining a temperature between 25° C. and 30° C. and a pH level between 6.0 and 7.0.

According to one exemplary embodiment of the present disclosure, the solid substrate fermentation method utilizes a mixture of agricultural by-products and supplements, including rice bran and wheat straw, to facilitate the growth and biomass production of Pseudomonas fluorescens strain CGA FPBPF-034.

According to one exemplary embodiment of the present disclosure, the application of the biofertilizer composition to the plants involves spraying onto foliage or root zones using conventional spraying equipment, facilitating even coverage and uptake by the plants.

### EXAMPLE—1

Process for isolation and cultivation of Pseudomonas fluorescens strain CGA FPBPF-034 from the rhizosphere: A method for isolating and cultivating Pseudomonas fluorescens from the rhizosphere of may include the steps of:
a. Selecting a suitable location where Pseudomonas fluorescens may be present, such as the rhizosphere of plants.
b. Collecting soil samples from the rhizosphere using a sterilized soil sampler or a trowel, ensuring samples are collected from the root zone of plants of interest.
c. Transferring the collected soil samples into sterile, airtight containers or bags to prevent contamination during transport.
d. In a sterile laboratory environment, aseptically weighing and recording the mass of the collected soil samples.
e. Preparing serial dilutions of the soil samples using a sterile diluent, with dilution factors varying depending on the expected microbial load, but commonly ranging from 10\hat{ }2 to 10\hat{ }6.
f. Plating aliquots of the diluted soil samples onto selective agar plates suitable for Pseudomonas fluorescens isolation, with Pseudomonas Specific Media (PSM) composition consisting of Tryptone 10 gm/L, Gelatin peptone 16 gm/L, Potassium sulfate 10 gm/L, Magnesium chloride, anhydrous 1.4 gm/L, Agar 15 gm/L, pH (at 25° C.) 7.1±0.2.
g. Incubating the agar plates at an appropriate temperature, typically around 28-30° C., for a duration of 24 to 48 hours or until bacterial colonies become visible.
h. Examining the agar plates for colonies exhibiting characteristics resembling Pseudomonas fluorescens, which typically manifest as small, fluorescent or non-fluorescent, rod-shaped bacteria.
i. Aseptically transferring individual Pseudomonas fluorescens colonies to fresh agar plates for the purpose of obtaining pure cultures, utilizing streak or loop techniques for isolation.
j. Conducting preliminary characterization tests to confirm the identity of the isolated colonies as Pseudomonas fluorescens, including Gram staining and fluorescent pigment production tests.
k. Preserving pure cultures of Pseudomonas fluorescens CGA FPBPF-034 through subculturing onto appropriate agar slants or cryopreserving them at −80° C. in suitable cryoprotectants.

### EXAMPLE—2

Molecular characterisation: Leveraging its diverse array of advantageous traits, the strain may potentially be subjected to molecular identification, which might entail DNA isolation, rDNA amplification at the 16S site using forward and reverse primers. The resulting amplified product may then be potentially subjected to partial sequencing services, which could be provided by MACROGEN, situated in Korea. The sequence data obtained may then potentially undergo comprehensive bioinformatics analysis, which may include meticulous examination through BLAST (Basic Local Alignment Search Tool) on the United States National Center for Biotechnology Information (NCBI) database. This analytical process may potentially lead to the conclusive confirmation of the organism's identity as Pseudomonas fluorescens. Subsequently, it may be worth noting that the accession code KY495220 was possibly assigned to the aforementioned strain following its retrieval from the NCBI repository.

### EXAMPLE—3

To conduct an in vitro experiment assessing the potential effectiveness of biocontrol against fungal pathogens such as Sphaceloma sp. and Cercospora sp., it is advisable to employ a systematic approach. This approach may encompass several critical steps, including obtaining pure cultures of the fungal pathogens, employing potential biocontrol agents, which may be in the form of commercial products or isolated antagonistic microorganisms, utilizing growth media tailored to each species, such as Potato Dextrose Agar (PDA), employing Petri dishes, utilizing sterilized water, setting up an incubator under appropriate temperature and lighting conditions, and employing precise measurement tools like rulers, calipers, or software applications for colony quantification.

The procedural sequence may potentially commence with the preparation of biocontrol agents, which may be conducted in accordance with manufacturer specifications or established laboratory protocols. In scenarios where antagonistic microorganisms are isolated, their cultivation within suitable growth media may be pursued. Subsequently, distinct pure cultures of Sphaceloma sp. and Cercospora sp. could potentially be obtained from infected plant tissues or alternative sources. Cultivation on growth media appropriate for each species may yield fresh spores essential for subsequent inoculation.

Simultaneously, petri dishes may be prepared using specific growth media tailored to the individual fungal pathogens, with the common choice being PDA. The introduction of the pathogens onto the petri dishes might involve aseptically transferring small quantities of spores from each respective culture, potentially using a sterile inoculation loop or needle. In the subsequent phase, the application of biocontrol agents may take place, and these agents could be introduced onto designated segments of the same petri dishes or individual dishes. Careful spatial separation may be maintained to potentially prevent any cross-contamination. Additionally, control plates may also be prepared concurrently, containing only the pathogen inoculum, strategically forming a basis for comparative assessment alongside the biocontrol treatments.

The prepared petri dishes may then be positioned within an incubator specifically configured to meet the growth requirements of each fungal pathogen. Systematic monitoring of the dishes may ensue, with the goal of closely observing fungal growth and developmental progress. The dimensions of fungal colonies may be meticulously assessed using precise measurement tools such as rulers, calipers, or specialized image analysis software. These measurements may be methodically recorded for each treatment throughout the designated time frame.

Upon data collection, a comprehensive statistical analysis may be undertaken, potentially employing methodologies such as ANOVA, to determine significant differences in growth suppression between the samples treated with the biocontrol agents and the control samples. These findings have the potential to yield valuable insights into the effectiveness of the biocontrol agents subjected to testing. The outcomes may suggest that the growth of pathogens was potentially significantly inhibited in cowpea, groundnut, and soybean seeds subjected to inoculation with Pseudomonas fluorescens, in comparison to the control group.

### EXAMPLE—4

Preparation of Mother Culture: The isolated strain might potentially undergo a subculture on PSM agar medium, which may then be incubated in a dark environment. After the incubation period, a pure culture of Pseudomonas fluorescens CGA FPBPF-034 may be introduced into a conical flask containing PSM Broth medium. This flask could then be placed in an incubator for a period of 3 days. Upon the conclusion of the incubation period, the cultivated cell biomass may be collected through centrifugation and potentially employed for the subsequent stage of fed-batch fermentation.

### EXAMPLE—5

Fed-batch fermentation may offer enhanced control over the fermentation process, potentially resulting in improved productivity, product quality, and the potential to customize formulations for specific applications. Consequently, it may be considered a valuable approach for preparing formulations across diverse industries, including biotechnology, pharmaceuticals, and food production. An organized approach for potentially carrying out fed-batch fermentation of Pseudomonas fluorescens could entail the following steps:
Step 1: Inoculum Preparation: Start with a well-characterized culture of Pseudomonas fluorescens strain CGA FPBPF-034. Inoculate a suitable seed medium and incubate for 16-24 hours to reach the mid-log phase.
Step 2: Bioreactor Setup: Sterilize the fermentation bioreactor and associated components. Prepare the fermentation medium with specific components.
Aseptically transfer the seed culture into the bioreactor.
Step 3: Fed-Batch Operation: Initiate fed-batch operation with controlled agitation, aeration, and continuous nutrient feeding. Monitor and control critical parameters such as pH, dissolved oxygen, agitation rate, and temperature. Adjust feed rate and nutrient concentrations as needed for optimal growth.
Step 4: Harvesting: Monitor the fermentation process until the desired cell density or product concentration is achieved. Harvest the culture by separating biomass from the fermentation broth using appropriate methods.
Step 5: Formulation Preparation: Further process the harvested biomass and fermentation broth, including cell disruption, extraction, and purification as required. Formulate the final product based on intended applications. Perform quality control and analytical testing on the formulated product.
Step 6: Packaging and Storage: Package the formulated product in suitable containers. Label containers with relevant information, including product details, batch number, expiration date, and usage instructions. Store the formulated product under appropriate conditions for stability and shelf life.

### EXAMPLE—6

Inoculation effect of Pseudomonas fluorescens formulation on cowpea, and groundnut seed emergence:
Materials Needed: Cowpea and groundnut seeds (50 seeds per treatment); Pseudomonas fluorescens formulation (laboratory-prepared, as illustrated in Example 2); Sterile water; Seed germination trays; Sterile potting mix or soil.
Seed Treatment: The foundational step involves dividing the seeds of each crop into groups consisting of 50 seeds for each treatment category, namely the inoculated and non-inoculated groups. The seeds designated for the inoculated treatment may be immersed in a prepared formulation of Pseudomonas fluorescens, potentially at a concentration of 2×106, allowing ample time, specifically around 60 minutes, for adherence. Conversely, an equivalent set of seeds for each crop may be kept as a control group without any treatment involving Pseudomonas fluorescens.
The germination trays or containers can be filled with sterile potting mix, establishing an appropriate environment for the seeds. Subsequently, 50 treated seeds and 50 control seeds for each crop may be evenly planted in separate rows within the germination trays. To maintain optimal moisture levels, a gentle watering of the seeds may be conducted. Each tray should be carefully labeled, indicating the crop type, treatment (inoculated or control), and date.
These germination trays are then positioned in a controlled environment, where suitable light and temperature conditions are possibly maintained for each crop. A daily record of the number of emerged seedlings for each treatment and control group may be conducted over a specified duration of 15 days. The data collected may subsequently be subjected to analysis in order to assess the impact of inoculation on seedling emergence. The results of the analysis may suggest that cowpea, groundnut, and soybean seeds, when inoculated with Pseudomonas fluorescens, could exhibit enhanced seed emergence, potentially ranging from 30% to 35%, when compared to the control group.

The impact of introducing Pseudomonas fluorescens into the ecosystem with the intention of addressing Scab disease in Cowpea (Vigna unguiculata) and late leaf spot disease in Groundnut (Arachis hypogea) may potentially be comprehensively assessed through rigorous field trials that span both the rainy and dry seasons. These trials might be carefully executed during the rainy season of 2016, encompassing the period from July to December, followed by a subsequent phase during the dry season of 2017, spanning from January to June. The trials may potentially be conducted at two distinct locations, with the utilization of Trichoderma harzianum as the selected biocontrol agent for both crops.

In another exemplary embodiment of this present disclosure, the procedures employed for planting in the field trials might encompass various approaches, including the potential use of a commercial seed planter. Subsequent application methods may include soil mixing (at a rate of 2.5 kg of formulation per 25 kg of soil per hectare, broadcasted during land preparation), seed treatment (at a rate of 10 g per kg of seed), and foliar application (at a rate of 5 g or 5 ml per liter of water at different time intervals: 15 days after seed treatment, 25 days after spraying, 45 days after spraying, and 60 days after spraying). These applications could potentially be carried out with handheld sprayers, aiming to ensure optimal dispersion at a rate of 250-300 liters per hectare.

A randomized complete block design (RCBD) may potentially be employed in all field trials, encompassing both cowpea and groundnut crops. Each individual crop trial may potentially cover a substantial area, extending to 10 acres. The experimental setup may involve the treatment of Pseudomonas fluorescens, in addition to two distinct control groups: one subjected to chemical fungicide control (Carbendazim: 2 Kg/ha at the specified intervals) and another left untreated as a control. This triple-replicated arrangement may be meticulously adopted to assess the incidence of diseases and the potential efficacy of biocontrol agents against pathogens. Upon reaching maturity, the crops might be harvested, followed by seed collection and cleaning, allowing for precise yield measurements. The enlightening results derived from these trials may potentially reveal a significant improvement in yield for both cowpea and groundnut crops when compared to their respective control groups.

Comprehensive and detailed findings of these results may be presented in Table 1 and Table 2, potentially confirming the substantial positive impact of introducing Pseudomonas fluorescens into the ecosystem on crop yield. These results might underscore the potential effectiveness of Pseudomonas fluorescens treatments in reducing disease incidence and enhancing agricultural productivity, thereby potentially making notable contributions to biocontrol and fostering abundant crop production.

TABLE 1
Effect of Pseudomonas fluorescens Inoculation on Cowpea disease
incidence, disease severity, grain yield.
Effect of Pseudomonas fluorescens Inoculation on Cowpea disease
incidence, disease severity, grain yield.

| Disease incidence (%) | | Disease severity (%) | | Grain yield (Kg/ha) | |
|---|---|---|---|---|---|
| Location-1 | Location-2 | Location-1 | Location-2 | Location-1 | Location-2 |

Rainy season

| | | | | | | |
|---|---|---|---|---|---|---|
| Pseudomonas | 40.81 c | 43.58 c | 17.10 b | 18.11 c | 2296.55 b | 1442.51 a |
| fluorescens | | | | | | |
| strain | | | | | | |
| CGA FPBPF-034 | | | | | | |
| Funguforce | 50.85 b | 53.88 b | 19.11 b | 22.13 b | 2585.25 a | 1197.06 b |
| (Carbendazim + | | | | | | |
| Mancozeb) | | | | | | |
| Untreated control | 95.61 a | 93.99 a | 40.72 a | 65.54 a | 1032.71 c | 792.20 c |
| CV | 1.9 | 6.1 | 4.3 | 2.8 | 2.6 | 8.4 |

Dry season

| | | | | | | |
|---|---|---|---|---|---|---|
| Pseudomonas | 9.05 b | 16.83 b | 6.04 b | 14.08 b | 1770.75 a | 1438.29 a |
| fluorescens | | | | | | |
| strain | | | | | | |
| CGA FPBPF-034 | | | | | | |
| Funguforce | 4.02 c | 11.07 c | 4.02 b | 11.07 b | 1437.88 b | 1265.97 b |
| (Carbendazim + | | | | | | |
| Mancozeb) | | | | | | |
| Untreated control | 11.27 a | 46.92 a | 11.90 a | 36.30 a | 1162.71 c | 872.47 c |
| CV | 4.4 | 5.6 | 14.1 | 7.4 | 2.8 | 2.7 |

Means followed by similar letters in a column are not significantly different at 5% level of probability according to least significance test,
CV: critical variance

TABLE 2
Effect of Pseudomonas fluorescens Inoculation on Groundnut disease incidence,
disease severity, grain yield.
Effect of Pseudomonas fluorescens Inoculation on Groundnut
disease incidence, disease severity, grain yield.

| Disease incidence (%) | | Disease severity (%) | | Grain yield (Kg/ha) | |
|---|---|---|---|---|---|
| Location-1 | Location-2 | Location-1 | Location-2 | Location-1 | Location-2 |

Rainy season

| | | | | | | |
|---|---|---|---|---|---|---|
| Pseudomonas | 15.09 b | 16.09 b | 17.10 b | 15.09 b | 1649.73 a | 1247.36 a |
| fluorescens | | | | | | |
| strain | | | | | | |
| CGA FPBPF-034 | | | | | | |
| Funguforce | 12.07 c | 14.08 c | 11.07 c | 10.06 c | 1378.13 b | 1074.34 b |
| (Carbendazim + | | | | | | |
| Mancozeb) | | | | | | |
| Untreated control | 55.33 a | 60.36 a | 32.19 a | 34.20 a | 1287.60 c | 784.63 c |
| | 2.5 | 2.5 | 2.2 | 2.3 | 2.1 | 2.1 |

Dry season

| | | | | | | |
|---|---|---|---|---|---|---|
| Pseudomonas | 19.11 b | 20.12 b | 16.09 b | 16.09 b | 1421.39 b | 1453.88 a |
| fluorescens | | | | | | |
| strain | | | | | | |
| CGA FPBPF-034 | | | | | | |
| Funguforce | 12.07 c | 15.09 c | 8.55 c | 10.23 c | 1498.54 a | 1388.29 b |
| (Carbendazim + | | | | | | |
| Mancozeb) | | | | | | |
| Untreated control | 45.27 a | 50.30 a | 31.18 a | 33.20 a | 1096.47 c | 996.58 c |
| | 2.3 | 2.3 | 2.3 | 2.3 | 2.1 | 2.1 |

Means followed by similar letters in a column are not significantly different at 5% level of probability according to least significance difference test.
CV: critical variance

### EXAMPLE—7

Viability test: The Pseudomonas fluorescens CGA FPBPF-034 may demonstrate the ability to control diseases and positively influence the germination and yield of diverse crop varieties. While certain microbes may exhibit beneficial characteristics for diverse crops, the existence of particular microbial populations within both plants and soil is chiefly influenced by factors such as substrate availability, prevalent environmental conditions (including soil moisture, pH, and organic matter content), and the competition among various microbial communities, without necessitating modification or supplementation. These advantageous microorganisms may potentially colonize plant roots, thereby improving nutrient uptake, producing growth hormones, and offering protection against diseases. This may foster an environment of enhanced plant growth and health.

Reference throughout this specification to “one embodiment”, “an embodiment”, or similar language means that a particular feature, structure, or characteristic described in connection with the embodiment is included in at least one embodiment of the present disclosure. Thus, appearances of the phrases “in one embodiment”, “in an embodiment” and similar language throughout this specification may, but do not necessarily, all refer to the same embodiment.

Although the present disclosure has been described in terms of certain preferred embodiments and illustrations thereof, other embodiments and modifications to preferred embodiments may be possible that are within the principles and spirit of the invention. The above descriptions and figures are therefore to be regarded as illustrative and not restrictive.

Thus the scope of the present disclosure is defined by the appended claims and includes both combinations and sub-combinations of the various features described hereinabove as well as variations and modifications thereof, which would occur to persons skilled in the art upon reading the foregoing description.

We claim:

1. A carrier-based agricultural biofertilizer composition, comprising:
an isolated Pseudomonas fluorescens strain CGA FPBPF-034 having the deposit accession number KY495220; and
an agriculturally acceptable solid or liquid formulation with an organic or inorganic stabilizer to provide stability and support for the Pseudomonas species, thereby enhancing the disease management efficacy and overall health of the plants.

2. The composition as claimed in claim 1, wherein Pseudomonas fluorescens strain CGA FPBPF-034 was isolated from rhizosphere and rhizoplane from Yola, Nigeria.

3. The composition as claimed in claim 1, wherein Pseudomonas fluorescens strain CGA FPBPF-034 was isolated by a serial dilution technique and spread plate method.

4. The composition as claimed in claim 1, wherein the agriculturally acceptable solid formulation comprises carriers selected from the group consisting of talc, vermiculite, peat, and compost, providing a suitable matrix for sustained release and growth of Pseudomonas fluorescens strain CGA FPBPF-034.

5. The composition as claimed in claim 1, wherein Pseudomonas fluorescens strain CGA FPBPF-034 has the ability to reduce the growth of fungal plant pathogens in vitro and can produce hydrolytic enzymes and Siderophore, thereby regulating plant disease and growth, improving root development, and enhancing overall plant vigor.

6. A method for preparing a carrier-based agricultural biofertilizer agricultural biofertilizer composition for enhanced disease management in legumes, comprising the steps of:
preparing a nutrient medium comprising suitable sources of carbon and nitrogen conducive to fungal growth;
inoculating said nutrient medium with a pure culture of Pseudomonas fluorescens strain CGA FPBPF-034, as identified by deposit accession number KY495220, and subjecting it to fermentation under controlled conditions;
extracting fungal spores and mycelia from the fermented broth utilizing appropriate separation techniques;
employing a fed batch fermentation technique to cultivate the fungal biomass to attain a concentration of 3.0×10\hat{ }8 colony forming units per gram (CFU/g);
diluting the fungal formulation to attain a desired microbial concentration of 3×10\hat{ }6 CFU per milliliter (CFU/mL) within a talc powder-based formulation or CFU/mL within a liquid formulation; and
administering the fungal formulation to the soil and applying it to the plants through suitable means, ensuring uniform distribution, thereby enhancing the vigor of the plants and augmenting the fertility of the soil, leading to improved disease resistance and management in leguminous crops.

7. The method as claimed in claim 6, wherein the nutrient medium comprises a combination of carbon and nitrogen sources, selected from the group consisting of sucrose, glucose, malt extract, and peptone, to provide an optimal growth environment for Pseudomonas fluorescens strain CGA FPBPF-034.

8. The method as claimed in claim 6, wherein the controlled fermentation conditions in step 2 comprise maintaining a temperature between 25° C. and 30° C. and a pH level between 6.0 and 7.0.

9. The method as claimed in claim 6, wherein the solid substrate fermentation method utilizes a mixture of agricultural by-products and supplements, including rice bran and wheat straw, to facilitate the growth and biomass production of Pseudomonas fluorescens strain CGA FPBPF-034.

10. The method as claimed in claim 6, wherein the application of the biofertilizer composition to the plants involves spraying onto foliage or root zones using conventional spraying equipment, facilitating even coverage and uptake by the plants.
